(19) United States
(12) Patent Application Publication
(10) Pub. No.: US 2014/0084179 A1
(43) Pub. Date: Mar. 27, 2014

(54) EXPOSURE CHAMBER AND A SYSTEM FOR REDUCTION OF PATHOGENS IN A BIOLOGICAL FLUID USING ULTRAVIOLET IRRADIATION BY LIGHT EMITTING DIODES

(71) Applicant: Hemalux Technologies LLC, Loveland, CO (US)

(72) Inventors: Ehud Ben-Hur, Loveland, CO (US); Heather Balji, Mahwah, NJ (US)

(73) Assignee: Hemalux Technologies LLC, Loveland, CO (US)

(21) Appl. No.: 13/975,419

(22) Filed: Aug. 26, 2013

Related U.S. Application Data
(63) Continuation-in-part of application No. 13/969,543, filed on Aug. 17, 2013.
(60) Provisional application No. 61/744,386, filed on Sep. 25, 2012.

(51) Int. Cl. A61L 2/00

(52) U.S. Cl. CPC A61L 2/0047
USPC 250/429; 250/435; 250/437

(57) ABSTRACT

An exposure chamber for reducing pathogens in a biological fluid such as whole blood or blood-derived products includes a serpentine-shaped UV-transparent flow path and closely positioned ultraviolet light emitting diodes configured for emanating UV irradiation towards the biological fluid at peak wavelength of 250 nm to 270 nm. The control system is provided to energize UV LEDs using various novel modes of modulating pulse width and current amplitude for LEDs so as to deliver higher UV intensity to the biological fluid but without overheating thereof or the LEDs.

### CROSS-REFERENCE DATA

This patent application is a continuation-in-part of the co-pending US patent application No. 13/969,543 filed 17 Aug. 2013; which in turn claims a priority date benefit from a related U.S. Provisional Patent Application No. 61/744,386 filed Sep. 25, 2012 by the same inventors and entitled “METHOD FOR PATHOGEN REDUCTION IN WHOLE BLOOD USING SHORT WAVELENGTH ULTRAVIOLET LIGHT”, both of which are incorporated herein in their respective entireties by reference.

### BACKGROUND OF THE INVENTION

The present invention relates to systems and methods for the UV-irradiation of a biological fluid for the purposes of reduction of pathogens therein. While the primary object of the invention is to treat blood, blood-based products and synthetic blood substitutes, the concepts of the present invention may be used for treating other fluids such as those encountered in beverage industries including dairy, distilling and brewing, as well as in water treatment industries including sewerage and purification systems.

The term “pathogens” is used broadly for the purposes of the present invention to include a variety of harmful microorganisms such as bacteria, viruses including among others a human immunodeficiency virus, a hepatitis A, B and C virus, parasites, molds, yeasts and other similar organisms which may be found in human or non-human blood and products derived from blood, as well as various other body fluids such (as for example milk) and synthetic fluids manufactured for use as replacements for any such body fluids or components thereof.

Blood transfusion in developed countries is very safe with regard to avoidance of transmitting of an infectious disease. This is primarily achieved by donor exclusion using questionnaires and screening for pathogens presence by means of serological methods and direct testing for nucleic acids. Despite these practices, there remains a risk of transmission of pathogens with the transfusion of cellular components of blood (such as red cells and platelets for example). This is at least in part because current screening tests leave a window of time after infection and before their sensitivity allows for detection of pathogens. In addition, screening does not takes place for rarely occurring pathogens or as yet unknown transmissible pathogens (Soland, E. M. et al. J. Am. Med. Assoc. 274: 1368-1373 (1995); Schreiber, G. B. et al. New Engl. J. Med. 334: 1685-1690 (1996); Valinsky, J. E. In: Blood Safety and Surveillance, Linden, J. V. and Bianco, C., Eds., Marcel Dekker, NY, 2001, pp. 185-219).

The use of pathogen reduction technologies has the potential of eliminating the remaining risks of transmission of infectious disease as a result of blood transfusion. Various approaches have been used to sterilize blood components (Ben-Hur, E. and B. Horowitz AIDS 10: 1183-1190 (1996); Ben-Hur, E. and R. P. Goodrich, In: Photodynamic Inactivation of Microbial Pathogens, Hamblin, M. R. and J. Gori, Eds. RSC Publishing, UK, 2011, pp. 233-263). The most promising methods are photochemical ones, two of which were approved by regulatory agencies for pathogen reduction in platelet concentrates. The Intercept method employs a psoralen and UVA light (Lin, L. et al. Transfusion 37: 423-435 (1997)) and the Mirasol method uses riboflavin and UVA+UVB light (Goodrich, R. P. et al. Transfusion Apheresis Sci. 35: 5-17 (2006)).

Short wavelengths ultraviolet light (UVC, 180-290 nm) is a known sterilizing agent that targets the nucleic acids of microorganisms (Setlow, R. B. and J. K. Setlow Proc. Natl. Acad. USA 48: 1250-1253 (1962)). It has been used for pathogen reduction in optically-transparent biological fluids such as plasma (Chin, S. et al. Blood 86: 4331-4336 (1995)) and is being studied also in platelet concentrates (Bashir, S. et al. Transfusion 53: 990-1000 (2013)). However, in opaque biological fluids such as red cell concentrates as well as in whole blood, UVC penetration is very limited due to absorption of UV irradiation by the red cells. As a result, all attempts to use UV irradiation for sterilizing whole blood or red cells have been unsuccessful so far.

Therefore, there is a need for an effective system and method for reducing pathogens in a biological fluid such as blood.

Attempts to irradiate blood or other opaque biological fluids with UV light have been described before. The exposure of a biological fluid to UV irradiation can result in damage to various components of the biological fluid, for example enzymes and other functional proteins. Therefore, the UV irradiation source should not be too powerful nor may the fluid be exposed to the UV radiation for too long, if one is to avoid damaging the components of the biological fluid.

To ensure that substantially all of the fluid receives a sufficient dose of UV radiation, it has been found that intensive mixing of the fluid to be treated during UV irradiation increases the efficiency of the irradiation process. A variety of devices that include static mixers placed in the fluid flow pathway have been proposed such as those described in U.S. Pat. Nos. 6,312,593; 7,175,808; US Pat. Application Publications 2004/0039325; 2006/0270960; or PCT publications WO1997046271; WO2000020045.

In addition to mixing, a sufficient intensity of the UV irradiation needs to be provided by a source of UV irradiation. Traditional devices used as such source included low pressure mercury UV lamps and xenon UV lamps. Such lamps have a number of disadvantages when used for the purposes of the present invention as they produce low level of UV output given the energy requirements, have large size, fragile and if broken represent an environmental hazard of mercury contamination. In addition, traditional UV lamps produce UV output over a broad range of UV wavelengths, some of which may be harmful to the biological fluid.

There is a need for a new exposure chamber and a new system for reducing pathogens in a biological fluid with improved source of UV irradiation and specifically with the ability to provide high intensity of UV light in a small physical size. There is also a need for a new source of UV irradiation to provide efficacious UV irradiation at desired peak wavelength with low energy consumption.

### SUMMARY OF THE INVENTION

Accordingly, it is an object of the present invention to overcome these and other drawbacks of the prior art by providing novel exposure chambers and novel systems for reduction of pathogens in a biological fluid by exposing the fluid to a sufficient dose of UV irradiation.

It is another object of the present invention to provide novel compact exposure chambers for effective UV irradiation of a biological fluid with desired peak wavelength of ultraviolet light

It is a further object of the present invention to provide novel systems for reduction of pathogens in the biological fluid having sufficient UV irradiation intensity to treat opaque biological fluids such as whole blood and blood products.

The system of the invention is designed for inactivation of pathogens in the biological fluid such as a unit of whole blood suitable for transfusion. The system includes a novel compact exposure chamber comprising a source of UV irradiation and a closely positioned adjacent flow path configured to expose the biological fluid to UV irradiation. The source of UV irradiation may be configured to emit ultraviolet light in the UVC range of wavelengths. In embodiments, UVC light may be used with a predetermined peak wavelength from about 250 nm to about 270 nm. The system may include a pump to propagate biological fluid such as a unit of whole blood shortly after its donation through a UV-transparent flow path of the exposure chamber, for example a serpentine-shaped tube. The flow path may include one or more static mixer elements to cause intermittent or continuous mixing of the biological fluid during its propagation through the flow path. The flow path may be configured for exposure of the fluid to UVC irradiation from a suitable source such as a plurality of ultraviolet light emitting diodes (UV LEDs)—at an appropriate power density that allows sufficient UVC dose to be impinged on the fluid such that sufficient inactivation of pathogens takes place. The treated fluid may then be propagated out of the exposure chamber and collected in a new storage bag and the disposable flow path may be discarded after use.

To assure adequate exposure to UV irradiation, the source of such irradiation may be selected to produce high intensity UV light at desired peak wavelengths. Consistent UV irradiation may be provided by a plurality of UV light emitting diodes or UV LEDs. In embodiments, rows of such UV LEDs may be mounted on a printed circuit board and placed on one or both sides of the flow path for a more complete exposure thereof.

UV LEDs may be powered continuously or intermittently to keep their temperature in the optimum range. Various energizing modes including pulse width modulation are described herein aimed at maximizing UV output of the LEDs without overheating thereof.

In embodiments, several arrays of LEDs may be used in an overlapping manner—while some LEDs are powered on, other LEDs are turned off so as to allow cooling. Staggered position and powering of several arrays of LEDs may be used to continuously expose the flow path of the system to UV irradiation.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is block-diagram of the system of the present invention;

FIG. 2 is a side view of the exposure chamber including the flow path and the source of UV irradiation according to some embodiments of the invention;

FIG. 3 is a front view of the serpentine-shaped flow path of the exposure chamber;

FIG. 4 is a front view of a panel of UV light emitting diodes forming together a source of UV irradiation of the exposure chamber according to some embodiments of the present invention;

FIG. 5 is chart showing continuous energizing of LEDs with constant current and voltage;

FIG. 6 shows one example of energizing LEDs using pulse width modulation;

FIG. 7 shows another example of energizing LEDs using pulse width modulation;

FIG. 8 shows yet another example of energizing LEDs using pulse width modulation;

FIG. 9 shows a further example of energizing LEDs using pulse width modulation;

FIG. 10 shows still further example of energizing LEDs using pulse width modulation;

FIG. 11 shows a front view of a panel of UV LEDs comprising two arrays of closely positioned LEDs, which is suitable for a staggered scheme of powering LEDs; and

FIG. 12 shows one embodiment of suitable LED power and control circuitry.

### DETAILED DESCRIPTION OF THE PREFERRED EMBODIMENT OF THE INVENTION

The following description sets forth various examples along with specific details to provide a thorough understanding of claimed subject matter. It will be understood by those skilled in the art, however that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

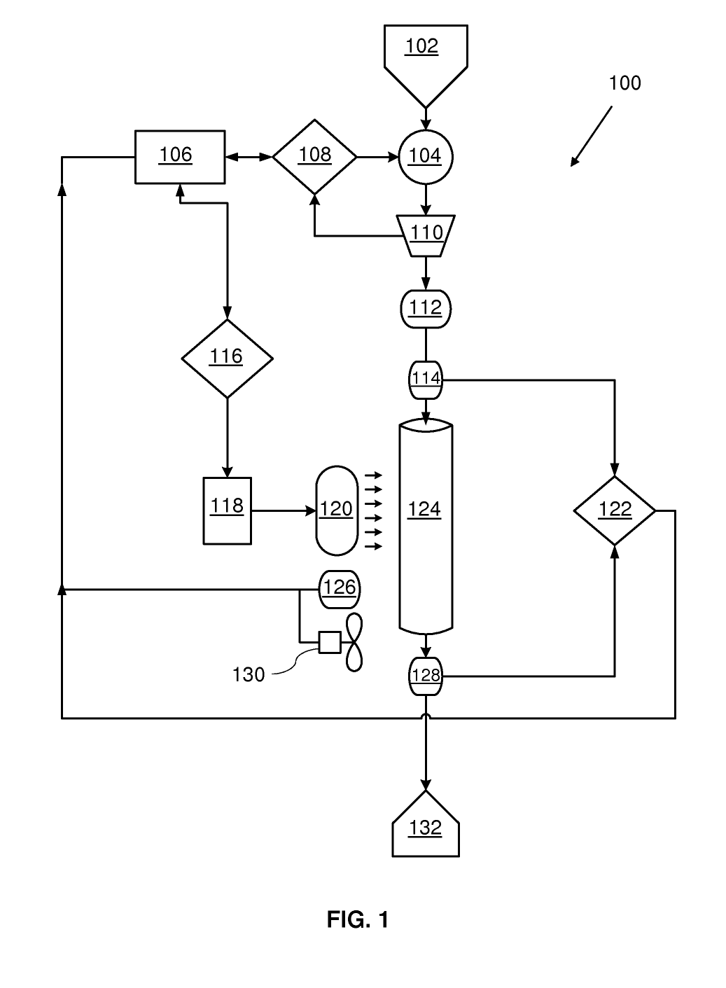

FIG. 1 shows a general block-diagram of the system 100 of the invention. A fluid supply source 102 may be used to draw the biological fluid from. Such fluid supply source may be a unit of blood collected from a donor, for example. Biological fluid may be drawn from or gravity fed into a pump 104 suitable for the purposes of pumping the biological fluid. A variety of pumps may be used for the purposes of the present invention. In the case of processing blood or blood products, a biocompatible atraumatic pump may be used such as a suitable peristaltic pump, centrifugal pump, diaphragm pump or another blood-compatible pump. The pump 104 may be controlled by a pump controller 108, which in turn may be operable by a central control unit 106. The pump controller 108 may be operated to cause the pump 104 to propagate the biological fluid through the exposure chamber with a desired constant or variable rate.

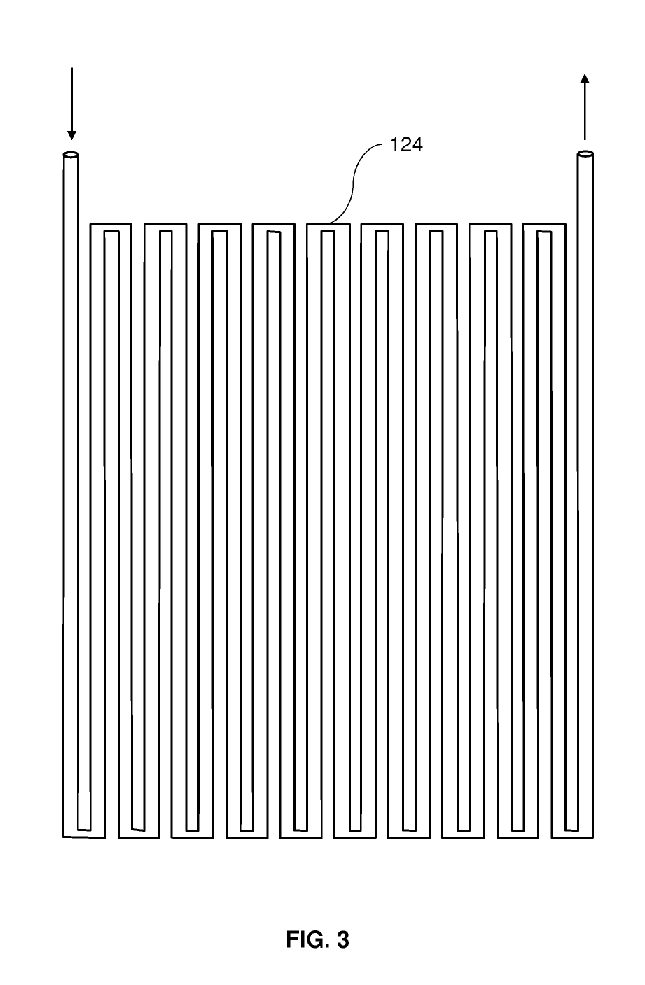

The biological fluid may be pumped by the pump 104 from the fluid supply 102 through the optional flow sensor 110 (operably connected to the pump controller 108), and further through an optional inlet pressure sensor 112 and an optional inlet temperature sensor 114 towards the exposure chamber, which includes a fluid flow path 124 described in greater detail below with reference to FIG. 3.

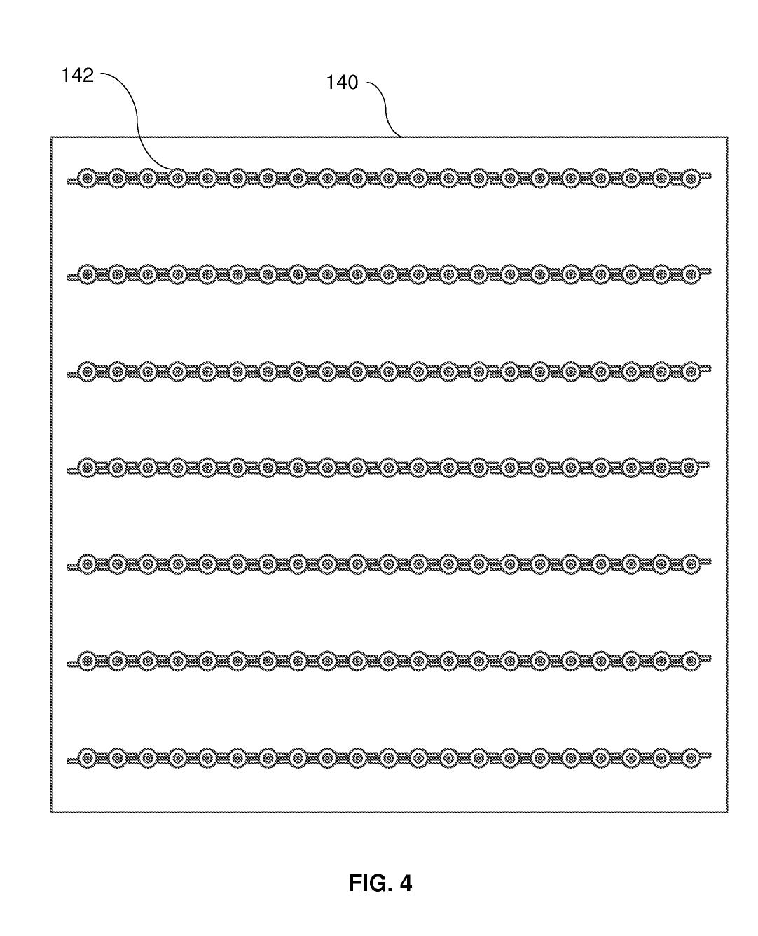

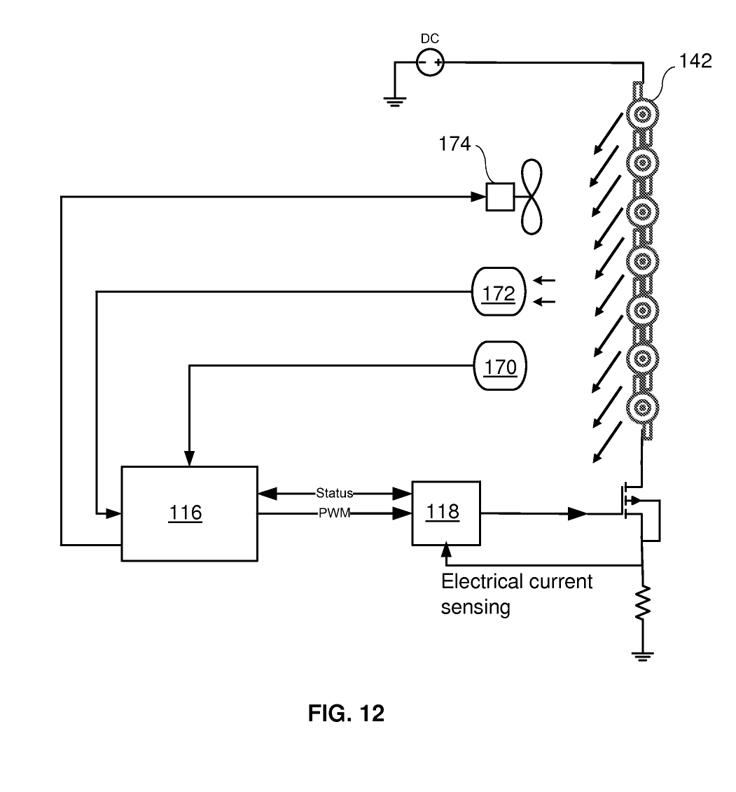

A UV source 120 may be used to provide UV irradiation suitable for reducing pathogens in the biological fluid while in the exposure chamber. A plurality of UV irradiation lights may form together the source of UV irradiation 120. Utilizing such plurality of lights as opposed to a single light allows a more uniform and spread-out UV exposure over a greater portion or preferably the entire flow path 124. The UV irradiation source 120 may be operated by a UV light source driver 118, which in turn may be controlled by the light control circuit 116 operable by the central control unit 106, better described below referring to FIG. 12. Further details of the UV irradiation source 120 are described below with reference to FIG. 4 through FIG. 11.

Following the exit from the flow path 124, the biological fluid may be directed through an optional temperature sensor 128 towards the outlet fluid collection element 132, such as a blood collection bag for example.

Additional elements of the system 100 may include a biological fluid temperature control system 122, which may be operably connected to the inlet 114 and outlet 128 fluid temperature sensors to detect a potential increase in fluid temperature above a predetermined threshold. Such temperature increase may be caused by too much energy passed into the biological fluid from the source of UV irradiation 120. Undesirable increase in temperature may also be caused by improperly slow rate of flow through the flow path 124, which may cause an over-exposure of the biological fluid to UV irradiation. In any case, the optional fluid temperature control system 122 may be connected to the central control unit 106 and used to at least trigger an alarm. In some embodiments, the system 122 may be used to activate or regulate the active cooling of the flow path 124 and the biological fluid contained therein.

The cooling of the UV irradiation source 120 and/or the flow path 124 may be accomplished in a number of known ways. Passive cooling may be accomplished by using a heat sink or by providing passive vents to expose the outer surface of the flow path to atmosphere. Active air cooling may be accomplished by providing one or more fans 130 or other cooling devices such as Peltier coolers thermally coupled to the heat sink, wherein such cooling devices may be activated upon the biological fluid or LEDs reaching an upper limit of allowable respectful safe temperatures. Liquid cooling systems may also be provided as an alternative to air cooling. Such liquid cooling systems may include circulation of a cooling liquid in thermal contact with LEDs 142 and/or the fluid flow path 124. When pumping blood or blood products, the safe upper temperature limit for the biological fluid may be set at 38 degrees C. For LEDs, the upper safe temperature limit may selected to be about 50 degrees C. (ideal operating temperature may be about room temperature, or 20 to 25 degrees C. while the maximum allowable temperature may not be above 100 degrees C.—higher operating temperature caused a significant reduction in UV output). The term “about” is used herein and throughout the specification to mean a deviation of +/−30% of the cited parameter.

In embodiments, the cooling elements of the system (such as cooling fans 130) may be activated for the entire duration of UV irradiating the biological fluid or for a portion thereof. The cooling elements may be activated on a predefined intermittent schedule or based on the feedback from the temperature sensors 114 and 128 or the optional temperature sensors configured to detect the temperature of the LEDs (not shown).

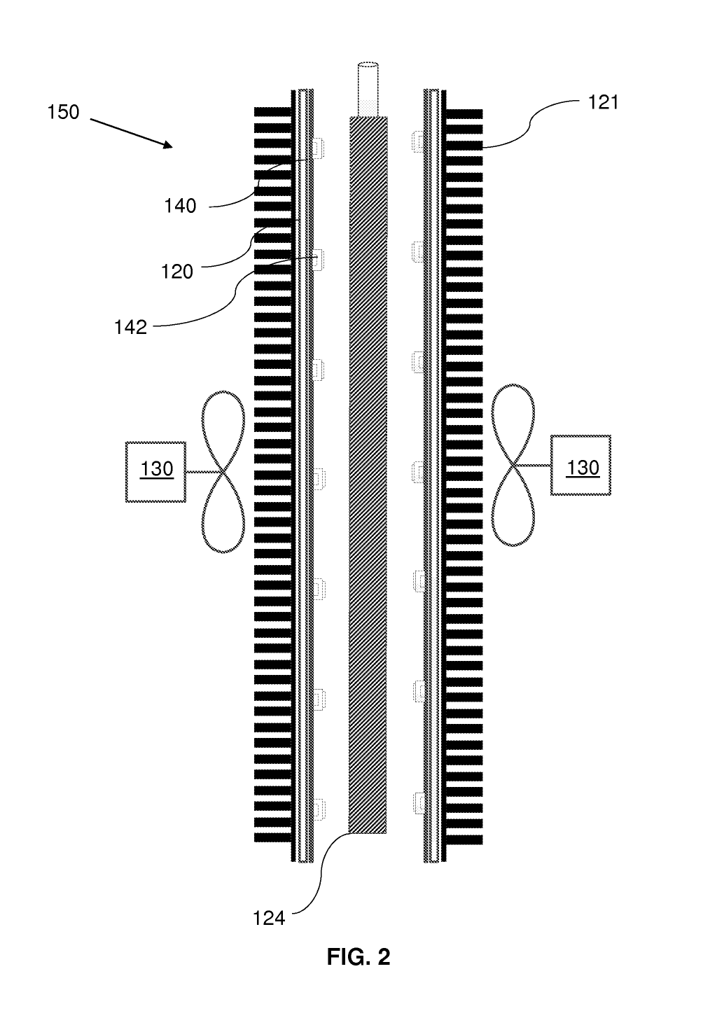

FIG. 2 shows a side view of the exposure chamber 150 of the system 100 according to the block-diagram in FIG. 1. Shown here are the flow path 124 and two UV sources 120 on both sides thereof, each UV source 120 comprising a printed circuit board (PCB) 140 with rows of surface mounted UV light emitting diodes 142 on one side and PCB heat sink 121 on the other side of the circuit board. Cooling fans 130 on one or both sides of the flow path 124 may be used to cool one or both the flow path 124 as well as the LEDs 142 to dissipate heat generated by the LEDs 142 during the time of their activation.

The details of the flow path 124 are better seen on the front view thereof shown in FIG. 3. The flow path may be selected to be sufficiently long to provide for sufficient UV exposure for the biological fluid propagating therethrough. The length of the flow path may vary from about 1 meter to about 20 meters. In embodiments, the length of the flow path may depend on the diameter thereof, the rate of biological fluid flow, the intensity of UV irradiation, the desired efficacy of pathogen reduction (“log kill” limit) and other factors. In embodiments, the length of the flow path 124 may be selected to be from about 4 meters to about 20 meters. In embodiments, the length of the flow path may be selected to be about 4 meters, about 6 meters, about 8 meters, about 10 meters, about 12 meters, about 14 meters, about 16 meters, about 18 meters, about 20 meters or any length in-between these numbers.

The cross-sectional shape of the flow path may be selected to be flat, oval, or round. In case of a round cross-sectional shape, the internal diameter of the flow path 124 may be selected to be from about 1 mm to about 8 mm. In embodiments, the internal diameter of the flow path 124 may be selected to be about 1 mm, about 2 mm, about 3 mm, about 4 mm, about 5 mm, about 6 mm, about 7 mm, about 8 mm, or any diameter in-between these numbers.

To provide a substantial length of the flow path in a small package, various methods of folding the flow path into a compact structure may be used. One such method is to form the flow path in a serpentine shape as shown in FIG. 3 with a well-defined inlet and outlet indicated by arrows in FIG. 3. The serpentine-shaped flow path 124 may be formed along a single flat plane so as to allow its exposure on both sides to the sources of UV irradiation 120 as shown in FIG. 2. Alternatively, the serpentine shape may be wrapped about a centrally-placed source of UV irradiation 120 or otherwise presented in a compact way to make the entire system easy to use.

In embodiments, all or some elements of the system 100 which are in direct contact with the biological fluid may be made disposable or reusable. In some embodiments, only the flow path 124 may be made disposable. In other embodiments, the flow path 124 along with the tubing for the peristaltic pump 104 as well as the necessary sensors or sensor access points may all be formed as a disposable cassette or a cartridge for easy handling before, during and after use. Such disposable cassette may have provisions to “plug into” the rest of the system including reusable UV irradiation source, the active portion (rollers) of the pump 104, and all the elements of the above described control system. Once assembled, the disposable portion of the system 100 may be attached at the inlet to the source 102 of biological fluid and at the outlet to the fluid collection element 132.

The serpentine-shaped flow path 124 may be made from a UV-transparent material such as glass, and in particular quartz glass. Alternatively, the flow path may be formed from plastics such as organic polymers, co-polymers and the like such as but not limited to cellulose products, PTFE, FEP, PVC and PE. In general, these materials have UV transmission properties in the range from 30 to 95% for a typical wall thickness, which may generally be between about 0.3 mm and about 2 mm.

FIG. 4 shows a front view of the source of UV irradiation 120 which may be a panel 140 such as a printed circuit board, which contains a plurality of UV irradiation light emitting diodes 142. Such elements may be arranged in rows and positioned next to the serpentine-shaped flow path 124 in the exposure chamber 150 as described above.

Using UV emitting LEDs in place of low pressure mercury lamps or other traditional sources of UV irradiation is a major point of innovation for the system of the present invention. UV emitting LEDs have a number of important advantages as explained below:
a. UV LEDs provide higher power density and UV irradiation intensity at a narrowly-defined predetermined peak wavelength, which is critical for effective operation of the system;
b. They can be instantly turned on and off without requiring the warm-up period;
c. They can be easily controlled using various energizing modes as described below—that allows fine adjustment of the amounts of photon energy delivered to the biological fluid;
d. They are physically small and can be densely packaged in close proximity to one another.

In addition, UV LEDs are advantageous for their durability, long life, energy efficiency (can be powered by a battery), environmental friendliness (no mercury), and safety (low voltage).

Generally speaking, LEDs may be fabricated by depositing very thin, highly crystalline layers (called epi-layers) of various compositions on a crystalline wafer substrate. The wavelength emitted by an LED is defined by the material properties of the epi-layers. The deposited atoms follow the crystalline template provided by the substrate, a process called epitaxial deposition. For most material systems, there is a native substrate. For example for diodes based on aluminum, indium, and/or gallium arsenide alloys, the native substrate is gallium arsenide (GaAs), whereas a silicon substrate may be used for diodes based on silicon.

Aluminum nitride (AlN) and gallium nitride (GaN) alloys may be used to fabricate ultraviolet LEDs emitting light with wavelengths in the germicidal range, which makes them suitable for the purposes of the present invention. Such LEDs may be fabricated on sapphire or aluminum nitride (AlN) substrates.

In embodiments, LEDs based on single crystal aluminum nitride (AlN) substrates may be deployed in the exposure chamber of the present invention. Such substrates may be used for growing active crystal layers of the LEDs with the low defect densities of the AlN substrate. These crystals (termed pseudomorphic epitaxial structures) exhibit sharp interfaces and smooth surfaces—with surface roughness of less than one nanometer. The resultant LED devices exhibit higher efficiencies and longer lifetimes in the 250-280 nm UVC wavelength range compared with light diodes fabricated from other technologies such as crystals grown on a sapphire substrate.

Surface-mounted AlN-based UV LEDs may therefore be advantageously used for the purposes of this invention. UV irradiation in the C range of wavelengths may be used. In particular, UV LEDs irradiating UV light in the range of wavelengths from about 240 nm to about 280 nm with a peak wavelength at about 250 nm to about 270 nm may be used. The light output of such LEDs may be about 20 mW, and a viewing angle is about 120°. High efficiency LEDs manufactured by Crystal IS (Green Island, N.Y.) may be used for the purposes of the present invention. These LEDs emit over 90% of their energy at the desired peak wavelength.

The peak wavelength selection is dictated by the need to be at or close to the absorption peak of nucleic acid which is 265 nm, which is critical for its destruction leading to inactivation of various pathogens and microorganisms.

Although UV LEDs are superior to other UV sources, they need to be energized with the proper understanding of their inherent performance. A simple way to operate the LEDs is to apply the nominal recommended electrical current at a nominal voltage to cause them to continuously emit UV light. Overdriving the LEDs at higher levels of electrical current may produce higher optical output at the beginning, which may drop sharply after sometime is elapsed due to overheating of the LED. Novel approaches of modulating pulse width and current level may provide for higher than nominal UV output of the LEDs without overheating thereof as described in more detail below.

In embodiments, two panels of LEDs may be positioned on both sides of the flow path 124. To further increase the efficacy of UV irradiation, the inner surface of the LED panels may be covered with a reflective material so as to redirect stray UV irradiation back towards the flow path 124. Examples of such reflective materials include highly polished aluminum, aluminum alloy mirror reflective surface, or poly-laminated aluminum foil, or metalized reflective films. The rate of biological fluid propagation through the flow path 124 may be selected such that the UV light dose absorbed by the fluid may be sufficient to reduce the level of pathogens or infectious agent contained therein. Maximum inactivation of pathogens is preferred but only up to a safe absorption level above which damage to the biological fluid may occur. For example, to minimize damage to the red cells, platelets and plasma proteins, a dose of 0.1 J/cm² of UVC light may be used.

In use, the system of the invention may be operated in the following way. Initial supply of the biological fluid may be positioned in the fluid supply source 102. The entire fluid contacting circuit as described above may be primed or filled with saline or the biological fluid itself may be pumped therethrough by the pump 104. The processing of the biological fluid may then be initiated by activating the source of UV irradiation 120 and the pump 104.

Pumping of the biological fluid through a serpentine-shaped flow path 124 causes it to be exposed to UV irradiation. Active mixing of the fluid is designed to bring pathogens to the surface of the flow and inactivate them by UV light. Flow mixing may be achieved for example by varying the flow through the pump 104 or by using static mixers in the flow path 124 as described in more detail in our parent patent application cited above.

Once the flow of the biological fluid is initiated by the pump 104, the LEDs 142 of the UV irradiation source 120 may be energized by the UV light driver 116 via the control circuit 118. The control circuit 118 may be programmed or otherwise configured to operate the driver 116 and energize the LEDs 142 using one of the following energizing modes.

In a first mode of energizing LEDs, a constant electrical current may be supplied to all LEDs as seen in FIG. 5. The level of the electrical current may be selected to be below, at, or above the recommended nominal operating electrical current. Higher than nominal electrical currents applied for shorter periods of time may be used to increase UV output of the LEDs provided sufficient cooling of the LEDs is available.

In embodiments, novel approaches to pulse width modulation of the electrical current may be used to increase the UV output of the LEDs. Pulse width modulation refers to periodic switching the power to the LEDs on and off. Pulse width modulation concept is known to be used to control LEDs, but this is done for the purposes of controlling dimming of the LED—by progressively reducing the duty cycle of the diode. The present invention provides a novel way to control LEDs—to increase their output as oppose to decreasing the output as describe in the prior art.

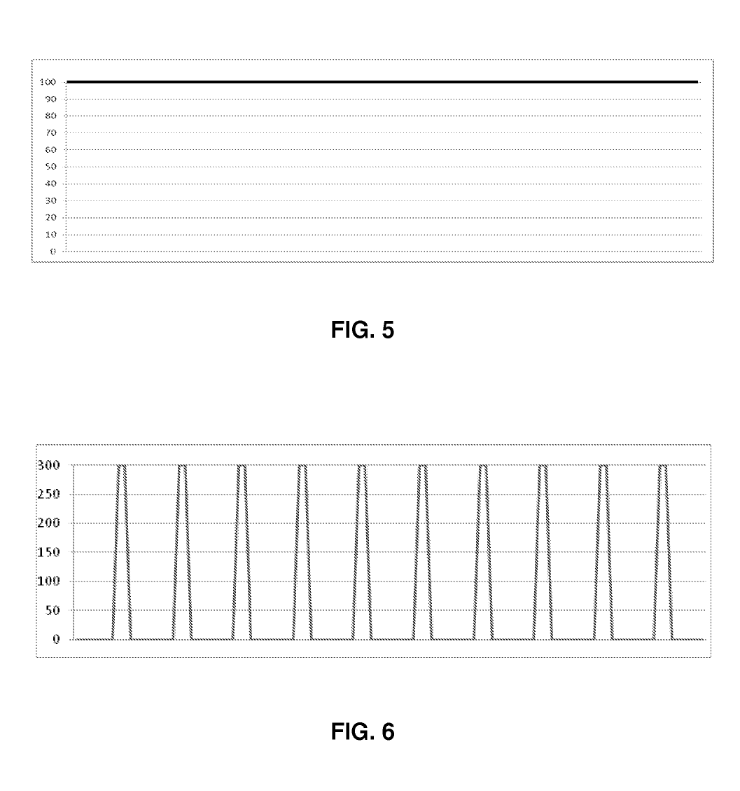

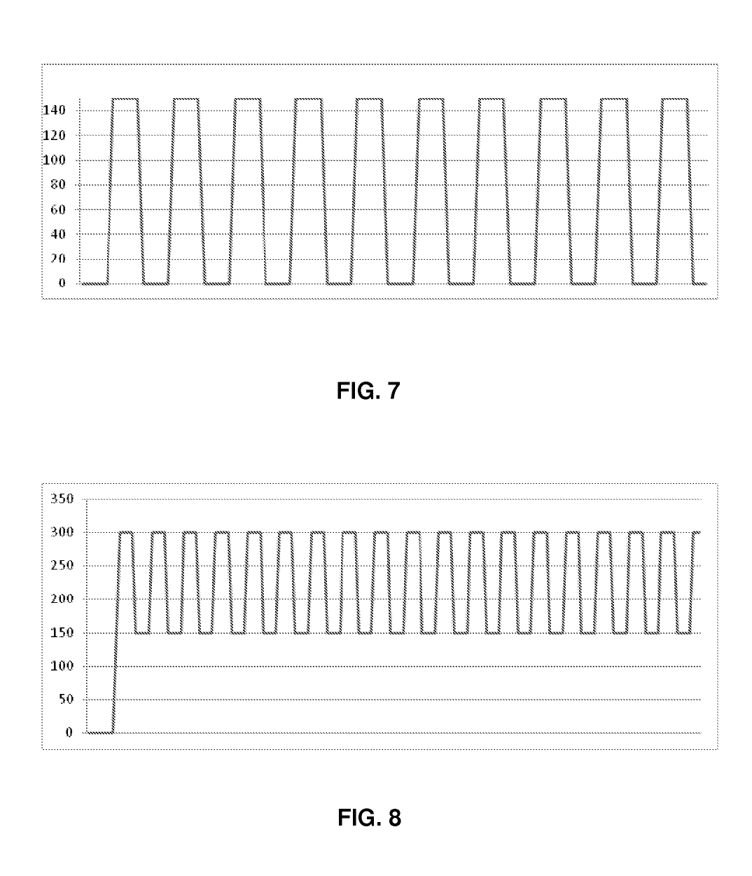

According to the present invention, the frequency of such pulse width modulation switching may vary from about 0.5 to about 5 kHz. In embodiments, such frequency may be about 2 kHz. The duty cycle of the pulse width modulation refers to the duration of powering the LED on as a percentage of the overall duration of the on-off cycle. In embodiments, the duty cycle may vary from about 1 percent to about 100 percent. In some embodiments, such duty cycle may be selected to be about 20 percent ON and 80 percent OFF as seen in FIG. 6. In other embodiments, the duty cycle may be selected to provide electrical current 50% ON and 50% OFF as seen in FIG. 7.

In embodiments, the duty cycle may be selected to be constant throughout the duration of processing the biological fluid within the exposure chamber 150. In other embodiments, the duty cycle may be variable. If variable duty cycle mode of LEDs energizing is selected, such variability may be done on a periodic basis, such as by following a predefined sinus waveform. In further embodiments, varying the duty cycle of the LED energizing may be tied to the reading of the temperature sensors for temperature of the biological fluid and/or the LEDs themselves. Detecting of overheating of either the biological fluid or the LEDs may be used to cause the control circuit 116 to operate the LED driver 118 with reduced duty cycle so as to allow for suitable cooling to take place. The system 100 may be configured to increase the total processing time if such overheating is detected and the UV intensity is reduced.

In embodiments, the peak electrical current (indicated as milliamps on the vertical axis of exemplary charts in FIGS. 5 through 10) may be selected to be fixed or variable. When fixed level of electrical current is selected, its value may be set at or above the recommended nominal value of electrical current for a given LED. Depending on the selected duty cycle, overdriving the LED from 1 to 1000 percent over the nominal electrical current may be selected. For shorter duty cycles higher levels of electrical currents may be selected. In exemplary charts shown in FIGS. 5 through 10, the nominal electrical current is selected to be 150 milliamps. Overdriving the LEDs to 300 milliamps for a duty cycle of 20% ON and 80% OFF at a frequency of 2 kHz may be suitable for the purposes of the present invention as seen in FIG. 6.

In further embodiments, the range of current modulation may be selected to be between a level above the recommended nominal electrical current to the level at or below the recommended nominal electrical current. One example of such energizing mode is shown in FIG. 8 showing a 50% duty cycle in which the electrical current is switched between a nominal exemplary value of 150 milliamps and the twice higher value of 300 milliamps. This approach allows for cooling of the LEDs while the current is at the nominal value while increasing the overall UV output while the electrical current is at the higher value. One advantage of such approach is that the LEDs are always ON and provide continuous UV irradiation to the biological fluid albeit with variable intensity.

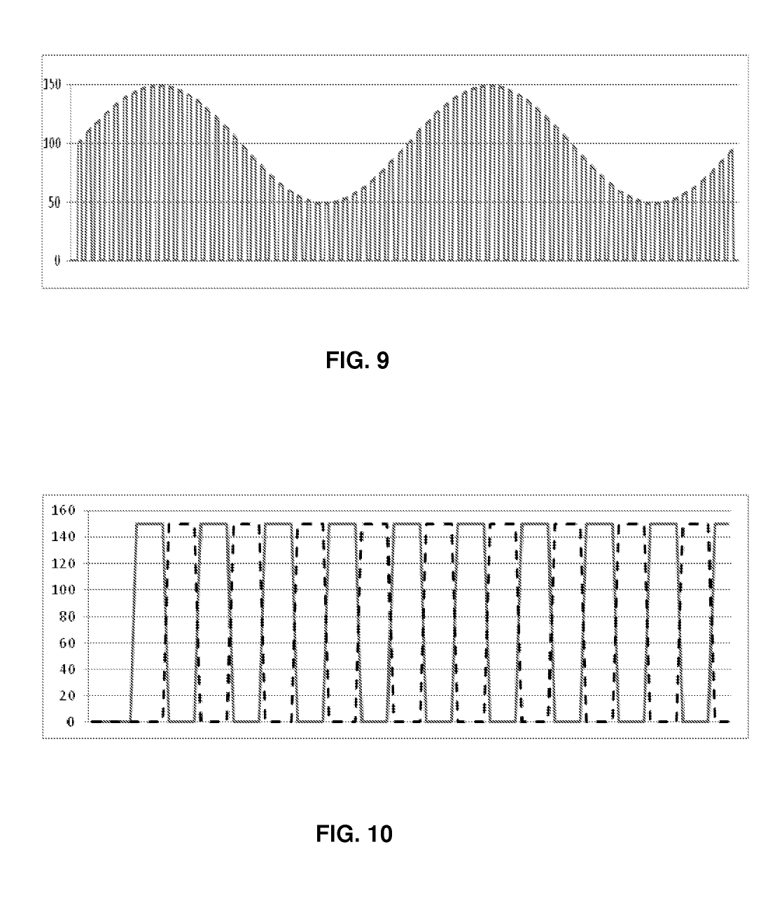

Further example of a novel mode of LED energizing is seen in FIG. 9 in which the pulse width may be modulated (for example at a 50% duty cycle) while at the same time the electric current amplitude may be varied for example using a sinus modulation waveform. In embodiments, a 120 Hz sinus modulating waveform may be used. In other embodiments, a range of the sinus waveform frequency may be selected from about 30 Hz to about 180 Hz, the modulation may be applied at fixed or variable frequency within this range. The electrical current amplitude may be modulated above and below the nominal recommended electrical current level or in other embodiments about the median level which may be set below or above the nominal recommended electrical current level. Modulating the electrical current amplitude about the current level above the nominal value—in combination with pulse width modulation may allow for overdriving the LEDs to produce higher UV output intensity but without the overheating of the LEDs or the biological fluid itself. Shorter duty cycle may be selected along with higher amplitudes of electrical current and vice versa. This mode of LED energizing also provides for higher UV output while never turning the LEDs off.

FIG. 12 shows one example of appropriate circuitry to drive LEDs 142 of the source of UV irradiation according to the present invention. Shown in FIG. 12 are a sample array of LEDs 142 powered with a suitable DC voltage to emanate UV irradiation (shown in solid diagonal arrows). Electrical current may be regulated by the control circuit 118, which may include for example a dynamic LED controller LM3463 by Texas Instrument Inc. (Dallas, Tex.). The circuit 118 may in turn be controlled by a driver 116, for example a microcontroller TMS320F28035 also by Texas Instruments Inc. The driver 116 may receive surveillance signals such as from an LED temperature sensor 170 and/or UV intensity light sensor 172. The driver 116 may be configured to activate the cooling system such as one or more fans 174 using one of the cooling control principles discussed above.

While some of the more straight forward energizing pulse width modulating modes described above may provide for increased UV output while the LEDs are turned ON at a higher than nominal electrical current, turning them OFF or substantially reducing the electrical current in the second portion of the pulse width modulation cycle causes the UV output to dramatically reduce or even be entirely off leaving the flow of the biological fluid propagating in the vicinity of a particular turned off LED underexposed or unexposed to UV irradiation. This may reduce the overall efficacy of the system and prompt to increase the length of the flow path 124 or compensate by propagating the same biological fluid through the exposure more than once.

To compensate for this limitation, further embodiments of the exposure chamber may be provided in which clusters of closely positioned LEDS may be used. Such clusters may include two, three or more LEDs positioned next to each other and configured to provide UV irradiation to the same portion of the flow path 124. In this case, activation of one LED of the cluster provides sufficient irradiation of the corresponding portion of the adjacent flow path while other LEDs may be turned off or activated with reduced levels of electrical currents. The energizing mode may include rotation of the full powered LED to switch to the next LED in a particular cluster such the initial LED may be turned down or off and allowed to cool, while the flow path is continuously illuminated with the appropriate UV at the sufficiently high level.

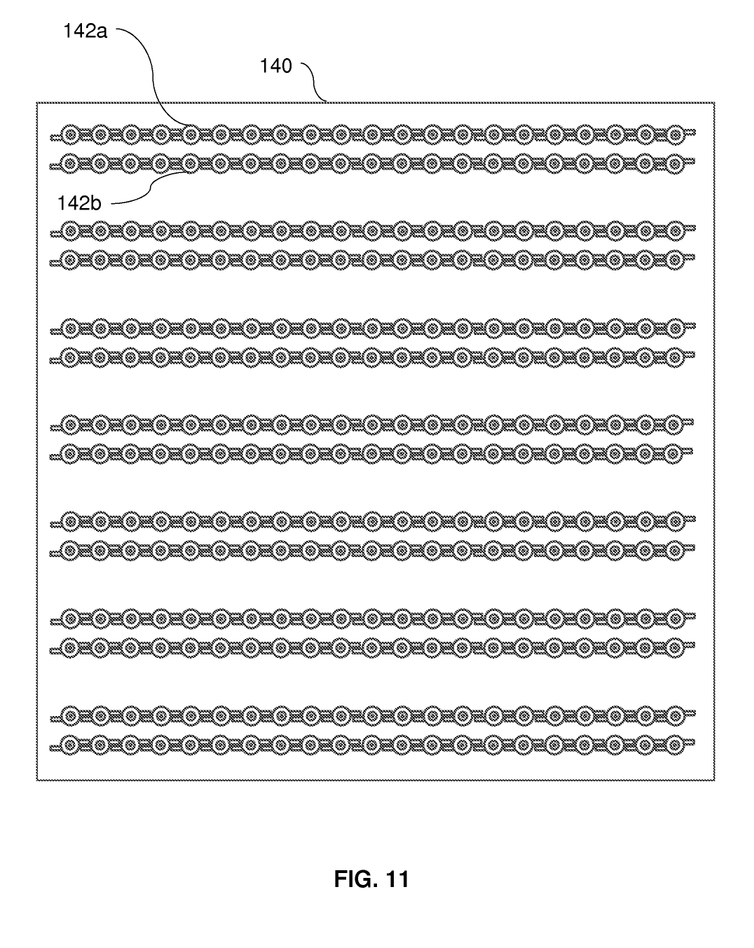

A physical example for such approach is illustrated in FIG. 11 in which a cluster of an LED 142a and 142b may be used to illuminate the same portion of the flow path 124. A row of LEDs 142a may be assembled close to the row of LEDs 142b. Activation of row 142a may be accomplished using a level of electrical current at or above the nominal value at an exemplary duty cycle of 50% as seen in FIG. 10 (solid line). That mode of activation may be alternated with activation of LEDs 142b at the same duty cycle of 50% and at the same level of electrical current, but with an appropriate shift in timing—see Fi. 10 dashed lines—designed to keep at least one of the LEDs 142a or 142b ON at all times. This mode of staggering the timing of turning LEDs ON and OFF keeps the flow path illuminated while allowing the LEDs to cool off during the down time.

More than two LEDs may be used in a single cluster. For example, three LEDs may be used with a staggered energizing of the duty cycle of 33% ON and 67% OFF—such that at least one LED is ON to provide for uninterrupted UV irradiation of the fluid path at higher IV intensity levels. In embodiments, more than three LEDs per cluster may be used. Higher number of LEDs allows to further shorten the duty cycle and increase the driving electrical current in this staggering energizing mode—and therefore increase the intensity of UV irradiation without overheating the LED assembly.

### EXAMPLE

An experiment was conducted to demonstrate the ability of a device constructed as described above to inactivate a model virus in blood. In this experiment, whole blood was spiked with bacteriophage φ6, which is similar in structure to HIV (the genome is RNA of about 7000 nucleotides and is lipid-enveloped). The final titer of virus in the blood was about 10 logs. The blood was pumped through the device at various flow rates and samples were withdrawn at each flow rate. The blood samples were diluted 10-fold with saline and centrifuged at 3,000 rpm. The supernatant was then assayed for virus titer after 10-fold serial dilutions, on a lawn of the host bacterium (Pseudomonas syringae), by scoring the number of plaques formed on the Petri dishes. The extent of virus inactivation (log₁₀) was calculated by comparing virus titer in treated blood with that of the control, untreated blood.

UVC power density during operation of the device was 2.2 mW/cm². The UVC light dose to which the blood was exposed was inversely related to the rate of blood flow, which varied from on average of about 5 ml/min to about 20 ml/min. The volume of the blood exposed to UVC inside the device was 100 ml. The transit time of the blood was therefore 20 min at 5 ml/min and 5 min at 20 ml/min. The total UVC dose calculated from the power density and transit time varied from 0.66 to 2.64 J/cm². The results are shown below:

| UVC light dose (J/cm²) | Virus inactivation (log10) |
|---|---|
| 0.66 | 1.2 |
| 0.99 | 1.5 |
| 1.32 | 2.0 |
| 2.64 | 3.2 |

These results demonstrate the ability of the device to inactivate over 99.9% of a model RNA virus in a UVC light dose-dependent manner.

The herein described subject matter sometimes illustrates different components or elements contained within, or connected with, different other components or elements. It is to be understood that such depicted architectures are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

Although the invention herein has been described with respect to particular embodiments, it is understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. An exposure chamber for reduction of pathogens in a biological fluid, the exposure chamber comprising:
a UV-transparent flow path for the biological fluid to be propagated therethrough, said flow path is configured to expose the propagating biological fluid to UV irradiation, and
a first plurality of spaced apart ultraviolet light emitting diodes positioned adjacent to said flow path so as to evenly deliver UV irradiation to the biological fluid when propagated along at least a portion of said flow path.

2. The exposure chamber as in claim 1, wherein said flow path is arranged in a serpentine configuration defining a first flat side, said first plurality of ultraviolet light emitting diodes closely located along said first flat side of said flow path, wherein upon activation of said first plurality of ultraviolet light emitting diodes, the biological fluid in said flow path is exposed to evenly distributed UV light irradiation from said first flat side.

3. The exposure chamber as in claim 2, wherein said serpentine-shaped flow path is defining a second flat side opposite said first flat side, said exposure chamber further comprising a second plurality of spaced apart ultraviolet light emitting diodes closely located along said second flat side of said flow path, wherein the biological fluid in said flow path is exposed to evenly distributed UV irradiation from both of said first flat side and said second flat side upon activation of all ultraviolet light emitting diodes.

4. The exposure chamber as in claim 3 further including at least one reflective surface configured to reflect UV light and located at least behind one of said first plurality or said second plurality of ultraviolet light emitting diodes, wherein said reflective surface is configured to increase UV light irradiation of the biological fluid emanating from said first plurality or said second plurality of ultraviolet light emitting diodes.

5. The exposure chamber as in claim 1, wherein said ultraviolet light emitting diodes are fabricated over single crystal aluminum nitride substrates.

6. The exposure chamber as in claim 1, wherein said ultraviolet light emitting diodes have a peak wavelength of the emitted ultraviolet light from about 250 nm to about 270 nm.

7. The exposure chamber as in claim 1 further comprising a cooling system configured to cool said flow path or said ultraviolet light emitting diodes.

8. The exposure chamber as in claim 7, wherein said cooling system is continuously or intermittently activated during the processing of the biological fluid based on a predetermined schedule.

9. The exposure chamber as in claim 7 further comprising a temperature sensor configured to detect a temperature or either said biological fluid or said ultraviolet light emitting diodes.

10. The exposure chamber as in claim 9, wherein said cooling system is activated when said temperature sensor detects a temperature of either said biological fluid or said ultraviolet light emitting diodes exceeding a corresponding predetermined safe threshold temperature for either said biological fluid or said ultraviolet light emitting diodes.

11. A system for reducing pathogens in a biological fluid, the system comprising:
an exposure chamber with a UV-transparent flow path for the biological fluid to be propagated therethrough, said flow path is configured to expose the propagating biological fluid to UV irradiation,
a plurality of spaced apart ultraviolet light emitting diodes positioned in close proximity to said flow path so as to evenly deliver UV irradiation to the biological fluid when propagated along at least a portion of said flow path, and
a control system for energizing said ultraviolet light emitting diodes, whereby upon energizing of the ultraviolet light emitting diodes by said control system, the biological fluid is irradiated by UV light causing reduction in said pathogens therein.

12. The system as in claim 11, wherein said control system is configured to periodically turn said ultraviolet light emitting diodes ON and OFF so as to maximize cumulative photon energy delivery to the biological fluid without overheating said ultraviolet light emitting diodes.

13. The system as in claim 12, wherein said control system is further configured to energize said ultraviolet light emitting diodes with electrical current amplitude exceeding a nominal level of electrical current for said ultraviolet emitting diodes using pulse width modulation with a range of duty cycle from about 1 percent to about 100 percent.

14. The system as in claim 13, wherein said control system is further configured to energize said ultraviolet light emitting diodes with variable amplitude electrical current applied thereto.

15. The system as in claim 14, wherein said control system is further configured to vary said electrical current amplitude above and below a predefined level of electrical current, said predefined level of electrical current selected to be at or above said nominal level of electrical current for said respective ultraviolet light emitting diodes.

16. The system as in claim 14, wherein said control system is further configured to periodically vary said electrical current amplitude from above said nominal level to about said nominal level of electrical current.

17. A system for reducing pathogens in a biological fluid, the system comprising:
an exposure chamber with a UV-transparent flow path for the biological fluid to be propagated therethrough, said flow path is configured to expose the propagating biological fluid to UV irradiation,
a plurality of spaced apart clusters of ultraviolet light emitting diodes positioned in close proximity to said flow path so as to evenly deliver UV irradiation to the biological fluid from at least one ultraviolet emitting light in each cluster when said fluid is propagated along at least a portion of said flow path, and
a control system for energizing said ultraviolet light emitting diodes, said control system is configured for energizing at least one of said light emitting diodes per one of said clusters so as to provide an uninterrupted UV irradiation to said biological fluid.

18. The system as in claim 17, wherein each of said cluster including at least two ultraviolet light emitting diodes.

19. The system as in claim 18, wherein said control system is configured to energize each of said ultraviolet emitting lights in said cluster on an alternating schedule, whereby when one of said ultraviolet emitting lights is turned ON by said control system, the other one of said ultraviolet emitting lights is turned OFF.

20. The system as in claim 19, wherein said control system is further configured to intermittently energize said ultraviolet emitting diodes with electrical current amplitude at or above a nominal electrical current level suitable for continuous energizing of said ultraviolet light emitting diodes without overheating thereof.
